(19) United States
(12) Patent Application Publication
(10) Pub. No.: US 2007/0092571 A1
(43) Pub. Date: Apr. 26, 2007

(54) Non-irritating formulation and method for the intradermal delivery of substances

(76) Inventor: Henry B. Schur, Ft. Lauderdale, FL (US)

Correspondence Address: Robert J. Van Der Wall;Gables One Tower, Suite 1275, 1320 South Dixie Highway, Coral Gables, FL, 33146, US

(21) Appl. No.: 11/256,838

(22) Filed: Oct. 25, 2005

(51) Int. Cl. A61K 36/889; A61K 36/736; A61K 36/886; A61K 36/899; A61K 36/48; A61K 9/14

(52) U.S. Cl. 424/486; 424/725; 424/727; 424/750; 424/757; 424/744; 424/735

(57) ABSTRACT

This invention describes a composition of matter to facilitate intra-dermal delivery of a great variety of active substances, especially including high molecular weight cosmicuticals. Also disclosed is a method of forming the composition of matter and applying topically to patients, especially using a cream or patch. The active substance is initially combined with a biopolymer, which combination is subsequently linked to a base formulation whereby the base formulation aids in the intra-dermal delivery of the active substance without the skin irritation limitations of other formulations and dermal transmitting methods. The use of a combination of natural herbal, vegetable and animal products combined in this invention with biopolymers permits molecules of higher molecule weight to penetrate into the skin in a bioactive form by passive delivery means. The invention includes a product for the improvement of skin health and wrinkle reduction.

### BACKGROUND OF THE INVENTION

1. Field of the Invention

The present invention relates to the field of a composition of matter for the intradermal delivery of biologically active/chemical substances without the skin irritation limits of other formulations. The use of natural herbal, vegetable and animal products combined in this invention with biopolymers has shown to permit molecules of higher molecule weight to penetrate the skin in a bioactive form by passive delivery means. It also relates to a method of delivery of biologically active/chemical substances that specifically include high molecular weight cosmetics and also cosmecuetical-active proteins and numerous other active substances through intradermal delivery. These include, but are not limited to, penta-peptides, collagen, elastin and cosmetic ingredients such vitamins, herbal compounds, hormones, chemicals, and the like.

2. Description of Prior Art

Historically, substances were presented to the body through the route of oral ingestion, nasal sprays, intravenously or by injection through or into the skin. Dermal application of substance destined for systemic use have also been used with some success where the molecule being delivered was of small size (<300 Daltons) and of appropriate solubility.

Intradermal cosmetic administration has been touted as a reliable means of achieving efficacious distribution of cosmetic preparations where other means of administration are either discontinuous, labor intensive or where other routes prevent absorption or create inactivation problems. Cosmetics which are applied to the skin are not absorbed into the dermal layers of the skin without the use of some form of penetrant or physical energy. Without this adjunct the resulting application is incomplete and non-uniform absorption of the cosmetic results. This leads to inconsistent and erratic intradermal levels of the active substances. In addition, the need for active periodic administration, i.e., three times a day, requires total compliance by the individual. Due to the aforementioned disadvantages and, last but not least, due to its non-invasive character, intradermal administration has recently become very popular.

Due to the skin dynamics as a living organ and the physical makeup of the skin layers the skin has been shown to behave as a complex barrier to the passage of both simple and complex molecules. The concept of a semi-permeable membrane which follows the physical laws of thermodynamics and concentration/diffusion gradients does not hold up in practice as the molecular size (weight) and configuration increases in both parameters. Additionally, biological factors enter into the complex requirements for a intradermally delivered active substance.

Some chemical/biological molecules are rendered inactive due to the concentration of specific antibodies to them that are resident in the skin. Others can and do cause local irritation which prevents their use based on a medical safety issue. Overcoming both the molecular size and substance irritation problem while maintaining active biological results within the dermis has been a principal goal of this invention.

Delivery of active substances into the skin layers to a point whereby the substance can be effective in achieving their desired result using a cream or “patch” device of which there are many designs known to those skilled in the arts or similarly by compounding the active ingredient into an appropriate carrier for direct application to the skin, i.e., cream, lotion, balm, gel, rub and/or ointment. All of these methods of delivering active ingredients to the skin have been used and are in common use today for delivering a variety of substances.

The current invention overcomes the foregoing and other barriers and allows for the intradermal delivery of high (>500 Daltons) molecular weight substances as well as for the use of low (<300 Daltons) molecular weight substances that heretofore were excluded from this mode of delivery due to factors of irritation and/or solubility.

The shortcomings of invasive (injectable) and traditional topical administration are obviated by intradermal application of the active. A cream and/or patch is routinely applied to an area of the skin and the active is continually absorbed into the skin. The upper layer of the epidermis (stratum corneum) was previously considered an impenetrable barrier in terms of cosmecuetical delivery. The advent of skin enhancers has vastly improved the administration of low molecular weight drugs.

The skin is particularly targeted as an area of the body that can benefit from cosmetic formulations to improve both its appearance and health. Intradermal penetration of these ingredients would substantially improve there effectiveness The utility of such a mode of administration has been promoted with the discovery and development of a group of compounds that promote intradermal/transdermal penetration of the various actives. Such compounds are known in the art as penetration enhancers or skin enhancers. They are generally characterized to be from the group of monovalent branched or unbranched aliphatic, cycloaliphatic or aromatic alcohols of 4-12 carbon atoms; cycloaliphatic or aromatic aldehydes or ketones of 4-10 carbon atoms, cycloalkanoyl amides of C 10-20 carbons, aliphatic, cycloaliphatic and aromatic esters, N,N-di-lower alkylsulfoxides, unsaturated oils, terpenes and glycol silicates.

These compounds and their specific activity as penetration enhancers, are more fully discussed in the text “Transdermal Delivery of Drugs, A. F. Kydonieus (ED) 1987, CRC Press, and in such patents as Fankhauser, U.S. Pat. No. 4,913,905, Heiber, U.S. Pat. No. 4,917,676, and Sinnreich, U.S. Pat. No. 5,032,403.

As a result of these penetration enhancers, almost any pharmacologically active substance, to some degree, can be administrated intradermally/transdermmaly. See, for example, such patents as Zaffaroni, U.S. Pat. No. 3,598,122, Zaffaroni, U.S. Pat. No. 3,598,123, Zaffaroni, U.S. Pat. No. 3,742,951, Zaffaroni, U.S. Pat. No. 3,797,494, Zaffaroni, U.S. Pat. No. 3,948,254, Bernstein, U.S. Pat. No. 4,284,444 and Etscorn, U.S. Pat. No. 4,597,961. Examples of such pharmacologically active substances include antibacterials such as the penicillins, tetracyclines, second and third generation cephalosporins, chloramphenicol sulfonamides, sedatives and/or hypnotics, such as barbiturates, carbromal, antitussives such as codeine and dextromethorphan, anti-anxiety drugs such as the benzodiazepines including diazepam, buspirone, psychostimulants such as imipramine, amitriptyline and other tricyclic antidepressants, anti-psychotic drugs and tranquilizers such as lithium, chlorpromazine and haloperidol, reserpine, thiopropazate, parkinsonism control agents such as bromotriptine, percolide, the anticholinergics including benzotropine, procyclidine, amantadine (also an antiviral), hormones and hormone antagonists and agonists, including adrenocorticosteroids, insulin, androgenic, steroids, estrogenic and pro-gestrogenic steroids, thyroxin and its agonist 5-FU (fluorouracil), tamoxifen, antipyretics and analgesics such as aspirin/acetaminophen and other non-steroidal anti-inflammatory drugs (NSAID), analgesics based on morphine, morphine antagonists, vasodilating agents such as nitroglycerine, isorbide dinitrate, alpha and beta-blockers and other cardioactive drugs, antimalarials, antihistamines and anticholinergics including atropine, hyoscyamine or methscopalomine (for motion sickness), weaning agents such as nicotine (for tobacco addiction), and antiasthmatic bronchodilators such as formoterol, and combinations of such pharmaceutical active substances.

Of course, while feasible, not all of these active substances have yet been completely tested for efficacy by intradermal administration but many are under vigorous scrutiny. Other active substances at this time are not economically viable for such administration, as the cost of full safety testing is too great for the specific number of patients involved.

It is noted, in particular, that high molecular weight proteins and peptides have not had many successes in terms of passive delivery with a minimum degree of irritation. Many of the attempts to deliver high molecular weight substances have been achieved mostly through aggressive means.

Johnson, U.S. Pat. No. 5,947,921, teaches and elucidates possible mechanisms of skin permeant enhancement using both chemical and physical means. Johnson also discusses the need for preventing skin irritation during intradermal drug delivery due to either/or penetrant chemicals or drug actives. It describes the use of sonophoresis as a means to provide delivery of proteins and peptides through the skin with the use of chemical enhancers. In addition, the sonophoresis may also rely on other aggressive assists such as mechanical or osmotic pressure, magnetic fields, electroporation, or iontophoresis.

D'Angelo, et al., U.S. Pat. No. 5,614,212, describes delivery of drugs ranging from 500 to 6000 Daltons and encapsulation of a drug in polyvinylpyrrolidone (PVP) in a microsphere composed of alginate and optionally a cross-linked alginate. D'Angelo '212 does not teach the use of PVP as a “conditioner” nor does it teach a pre-use incubation phase whereby PVP acts as a “binding” agent to allow the active to be incorporated into a suitable formula for the non-irritating delivery of active components. The present invention improves and expands in new art the use of PVP as an excipient and in unanticipated ways over D'Angelo '212, in combination with other excipients to achieve a non-encapsulated, non-irritating delivery system. D'Angelo '212 also excludes PVP as the preferred enhancer. The present invention teaches that PVP is compatible with cosmetic actives when used in the stated new method elucidated herein.

D'Angelo, et al., U.S. Pat. No. 6,024,975 describes a way to deliver high molecular weight drugs by transdermal administration, consisting essentially of a drug having a molecular weight ranging from 50 Daltons to 25,000 Daltons, a polymer which is polyvinylpyrrolidone and an optional gelling agent. The patent claims the delivery of Calcitonin and Insulin and one that can be achieved by optionally adding electronic means to enhance absorption, microspheres and solubility enhancers chosen from a group including acetamide, N,N-dimethylacetamide, N,N-diethylacetamide, C.sub.10-C.sub.20 alkanoylamides, 1-N-C.sub.10-C.sub.20-alkylazacycloheptan-2-one, N-2-hydroxyethylacetamid-e, dimethyl sulfoxide, salicylates, polyalkylene glycol silicates, and mixtures thereof. In similar manner to D'Angelo '212, D'Angelo (975 does not teach the use of PVP as a “conditioner” nor does it teach a pre-use incubation phase whereby PVP acts as a “binding” agent to allow the active drug to be incorporated into a suitable formula for the non-irritating delivery of active components. The present invention improves and expands in new art the use of PVP/solvent replacement treatment as an excipient and in unanticipated ways over D'Angelo '975, in combination with other excipients to achieve a non-encapsulated, non-irritating delivery system.

Gertner, U.S. Pat. No. 5,707,641, teaches a pre-treatment step for insulin which consists of allowing the insulin hexamer to dissociate into a dimer or monomer over a 30 day period at temperatures over 4.degree. C. and preferably over 20.degree. C. This step effectively reduces the molecular weight from approximately 6000 to 3000-4000 Daltons and thus make its delivery art similar to many others in the field who have shown transdermal delivery of molecules below 4000 Daltons. The present invention does not rely on the reduction of the molecular weight of the drug active to achieve systemic delivery through the skin. The present invention teaches a new method of incubation of the active drug with a compound that will act as a “combining” agent and allow for the drug to attach to the excipient/penetrant formulation regardless of molecular weight. Gertner does not teach or suggest the addition of any compound to the active drug during his decomposition stage. The present invention utilizes an incubation period to allow the gentle combining of the “coupling” agent with the active drug which is carried out in a short time period (7 days) and can be accomplished at 4.degree. C. for product stability. Further, those skilled in the arts will appreciate the present invention's ability to be adapted to a wide variety of compounds that by their nature will not de-polymerize upon standing as a way to lower their molecular weight. In fact many drugs if put through the Gertner process would lose their efficacy.

Foldvari, U.S. Pat. No. 5,718,914, broadly describes the delivery of topical agents through the use of liposomes. The liposomes are described to be particulates able to pass through membranes having pores of 0.1 to 500 microns. The formulations are intended to be delivered through a patch in a reservoir behind the above described membrane. It also teaches the construction of a suitable patch to contain a composition of matter instant to the present invention along with patent literature references for same all of which are incorporated herein by reference.

Skinner, U.S. Pat. No. 5,449,670, teaches that there may be a “conditioner” effect in using some pyrrolidone compounds to aid in the delivery of active components below the 4000 molecular weight and teaches that preferably the molecular weight should be below 3500 molecular weight. The present invention teaches that specifically a vinyl pyrrolidone when incubated at specified conditions with an active compound can, when further formulated into the present invention, deliver drug compounds in excess of 5000 molecular weight. This being a great improvement over Skinner in that most new therapeutic drugs being developed are of large molecular weights (i.e., synthetic insulin, growth hormone, etc.). The present invention advances Skinner and teaches a new method and formula for using PVP in a heretofore unanticipated way even by those skilled in the arts.

Clement, U.S. Pat. No. 5,208,028, teaches the use of a multi-step emulsion mixture process. It uses particulates created from a combination of aqueous dispersion of fatty acids, fatty alcohols, oils, basic compounds such as triethoxylamine, saccharides, alginates, chitin, metal salts, structural polymers such as carboxypropyl cellulose or xanthan gums. All the combined components are emulsified into an aqueous dispersion which is then used for topical administration of cosmetic ingredients. However the present invention is not dependent on the cross linking of the Clement emulsion to achieve its result. Further Clement requires that “capsules” be formed to protect or isolate the active component prior to use. The present invention is an improvement over Clement as no “activation” is required and a mechanical dispenser (pump) is not required for the product to achieve its stated goal.

Ghosh, U.S. Pat. No. 5,431,924, teaches the fractionation of emu oil into a biologically active substance having claimed therapeutic value. Furthermore, it is claimed that to obtain this value the product must be placed in a suitable carrier for transdermal delivery. The present invention teaches that emu oil can be used in its unfractionated state as an excipient and protectorant of active pharmaceutical ingredients. The use of emu oil as an example of a refined avian oil for an excipient in the compounding of a transdermal delivery system is a new and significant advantage over previous saturated fatty acid emulsification excipients. The present invention, on the other hand teaches and claims that the use of emu oil in a new and novel emulsification/transport material when combined with other natural oils effectively aids in the transport of active drugs across the dermis while reducing inflammation at the application site. Ghosh does not teach the use of emu oil as a transport vehicle or as an emollient or as a humectant, all properties that are utilized in the present invention as an aid in the non-irritation delivery of active ingredients.

Fein, et al., U.S. Pat. No. 5,472,713, describes a method of lowering cholesterol or triglycerides through the oral, parenteral, enteral, rectal and systemic administration of 2-10 mls of emu oil per day.

SUMMARY OF THE INVENTION

Bearing the mind the foregoing, a principal object of the present invention is to provide a composition of matter comprising an active substance combined with a biopolymer, which combination is linked to a base formulation whereby the base formulation aids in the intradermal delivery of the active substance.

A further object of the invention is to utilize a composition of matter wherein this composition may be applied to the skin as a topical treatment such as a cream, lotion, balm, gel, rub and/or ointment.

Another object of the invention is the use of a combination of biopolymers and natural herbal and animal products in a composition of matter to intradermally deliver substances without irritation.

A related object of invention is to specifically include in the group of active substances to be administered not only cosmetic ingredients such as collagen and elastin, but vitamins, herbal compounds, hormones, chemicals, and the like.

Another principal object of the invention is to provide a viable system and method for the intradermal administration of active substances including high molecular weight substances, of upward of 150 Daltons with a polymer skin enhancer and an ingredient that minimizes inflammation.

It is a related object of the present invention to provide a method of intradermally administering an active substance that may be a high molecular weight drug, which in summary includes applying to skin of a patient a polymer skin enhancer, and applying to the skin of the patient an active (15% or more of the system) having a molecular weight of above 150 Daltons and preferably above 500 Daltons. The preferred skin enhancer is polyvinylpyrrolidone.

Another object of the invention is pre-incubation of an active substance, such as (but not limited to a high molecular weight active) with a biopolymer under conditions which are suitable for the mutual solubilization of the active substance and the polymer while maintaining the desired biological activity of the active substance.

Another object of the invention is for the composition to be applied using a intradermal patch.

A similar object of the invention is to provide a process whereby a biopolymer is combined with an active substance to be delivered and then linked to a base formulation which aids in the delivery of said active substance.

Another object of the invention is the combination of biopolymer and base formulation which prevents skin irritation caused by the active substance in the present composition of matter and method.

A further object of the invention is a system of adjusting the hydrophobic/lipophobic nature of the inventive composition of matter to allow various solubility of active substance with which it is intended to be used in the method of the present invention.

An additional related object of the inventive method is to provide that biopolymer is incubated with active substance to initiate binding reaction prior to combination with other ingredients.

One more object of the invention is to provide that a base formulation consisting of both aqueous and non-aqueous components is combined into a homogeneous mixture with adjustable viscosity.

A further object of the invention is the use of an organic solvent of high volatility in the preparation of the base formula that evaporates off during the homogenizing process.

Another object of the invention is to select the biopolymer from the group of polymers represented by polyvinylpyrroliodone, alginates, chitin, collagen, Elastin and similar materials.

An additional object of the invention is to include aloe vera in the group from which the herbal extract is selected.

A further object of the invention is to select the vegetable component from the group including the natural extract of: marcrocystis sp., oil of coconut, corn oil, soy oil, almond oil, and the like.

One more object of the invention is to select the natural animal products from the group that includes the oil from the avian species: emu, chicken, turkey, ostrich, glycerol, etc.

A further object of the invention is to include in the acceptable solvents ethyl alcohol, isopropyl alcohol, acetone, and methanol.

Other objects and advantages will be apparent to those skilled in the art upon review of the following descriptions and the appended claims.

In accordance with a principal aspect of the present invention, there are disclosed compositions of matter that include a polymer system for effecting delivery of high molecular weight active substances by intradermal administration. The system includes at least 15% by weight of an active substance having a molecular weight of more than 150 Daltons, a polymer which is preferably polyvinylpyrrolidone, a weight of the polymer being 7 to 35% by weight of the active substance, and an optional gelling agent having from 0 to 20% by volume of the system. The gelling agent can be chosen from the group consisting of alginates, chitin, collagen, elastin and the like.

The active substance of the polymer system may be a penta-peptide, i.e., ciruimide, collagen, or elastin. The polymer may be a biocompatible polymer of the pyrrolidone group, e.g., polyvinylpyrrolidone (PVP). If PVP is used it may have a K-value of K-10 or K-40. Other polymers with solubility characteristics similar to polyvinylpyrrolidone may also be considered.

The ingredient that is capable of inhibiting inflammation is obtained from the natural animal products consisting of the oil from the avian species such as emu, chicken, turkey, or ostrich and can represent anywhere from 1 to 20% of the composition, most preferably with the ingredient approaching 8-10% of the total composition.

The finished composition can be applied as a topical treatment wherein the compound is a unit dose dispensed from a suitable package and spread on the skin or alternatively can be incorporated into a intradermal delivery patch of a standard design and known to those familiar with the art and then applied to a selected area of the body. The composition may be fashioned as a intradermal patch, cream, lotion, balm, gel, rub and/or ointment. Topical use may be applicable in animals where a physical patch would be difficult to maintain in contact with the skin.

In accordance with another major aspect of the present invention there is provided a method that includes pre-incubation of an active substance, such as (but not limited to, a high molecular weight cosmecuetical) with a biopolymer under conditions which are suitable for the mutual solubilization of the active substance and the polymer while maintaining the desired biological activity of the said substance. This pre-incubation period is a required step to assure that the interaction of the active substance and the polymer have occurred. This step can be done at temperatures between 4 degrees Celsius and 37 degrees Celsius for times ranging from a few minutes to as much as 30 days.

The pre-imbibed polymer is then added to a solvent rich formulation and homogenized in a suitable high shear homogenizer during which the solvent is removed through appropriate means. The solvent used for this can is one that preferably is volatile and can be chosen from the following group such as ethyl alcohol, isopropyl alcohol, acetone, or methanol. The solvent rich formulation can contain solvents that are polar and non-polar, aqueous and non-aqueous or any combination thereof. The selection of solvent systems will vary as the chemical/physical properties of the substance to be delivered are varied. In the example cited an ethanol/water solvent system is used.

The viscosity of the finished formulation is adjusted during mixing to produce the desired consistency for the intended use. Since the formulation exhibits classic thixotropic properties, rest time must be incorporated into the process to avoid viscosity variations.

The non-aqueous components of the formulation comprises an emulsification of the carrier ingredients by first incorporating the non-aqueous soluble components with the biopolymer under high shear mixing and then adding the aqueous materials again under high shear mixing. Some of the non-aqueous components can be chosen from the group consisting of macrocystis sp., oil of coconut, corn oil, soy oil, or almond oil.

The percentage of active compound required will vary depending on the pharmocodynamics, delivery rate, solubility, dose requirements, bioavailability, and other factors. Active materials can represent from as low as 0.01% to as high as 60% of the total composition. Compounds with molecular weights from the low, i.e., 150-180 Daltons, to as high as many thousand Daltons, i.e., 6000 Daltons, can be used with this delivery system. The upper limit on molecular weight has not been established but can theoretically be as high as 25,000 Daltons.

### DETAILED DESCRIPTION OF THE PREFERRED EMBODIMENT

As required, detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the claims hereto appended and as a representative basis for teaching one skilled in the art to variously employ the present invention in virtually any appropriate circumstance.

The preferred embodiment of this invention has been derived from both animal experimentation and human clinical experimentation. A number of specific examples are presented hereinafter based on the results using curuimides.

Active ingredient skin irritability must also be considered when selecting the biopolymer for first step incubation which determined the selection of polyvinylpyrrolidone (PVP) as the biopolymer best able to act as linking/coupling agent and protecting agent for this process.

Part I of the process includes pre-manufacture of several of the components.

The PVP base used in Part II is made by dissolving PVP K-40 (Sigma Chemicals, U.S.) in an ethyl alcohol solution. A 25% concentration of the PVP is dissolved in 95% ethyl alcohol (VWR Scientific, U.S.) using high shear mixing to start and then continuing to mix at low speed for 24 hours. Precautions must be taken to avoid solvent evaporation while making this ingredient. After mixing and solution is complete the mixture must “rest” for an additional 24 hours to allow the compound to stabilize. Part “A”.

The Kelgin HV (Monsanto, U.S.) is prepared by slowly adding the powdered Kelgin HV to distilled water to make a 3% solution using a high shear mixer. The temperature of the product must not exceed 45 C. during the mixing process. The addition of methylparaben (1:10,000) to the Kelgin mixture will assure its biological safety and will act as a safe and effective preservative for the final product. The Kelgin mixture should stand at room temperature for 72 hours to allow for complete hydration of the polymer and stabilization. Part “B”.

Hyaluronic acid powder (Pentapharm, Basel Switzerland) is dissolved in a natural aloe vera extract (Lilly of the Desert, Denton, Tex.) so as to achieve a final concentration of 0.5% of the production lot. This must be done using high shear mixing while maintaining a temperature of 4-10° C. This mixture must incubate for 24 hours. Part “C”.

Part “D” is comprised of the following ingredients that are blended together with high shear mixing to produce a smooth uniform mixture blend: purified water, stearyl alcohol, cetyl esters wax, glyceryl monosterate, polyoxyethylene stearyl ether, sorbitol, isopropyl palmitate, methylparaben, propylparaben, captan.

Preparation of the final composition must proceed in such a manner as to preserve the purity and efficacy of the compound by using aseptic techniques throughout.

The final composition of compound is made as follows:

To the required calculated percentage of part “A” previously made, add the K-oil stock solution (Texas EMU Cooperative, U.S.) which has been stored at 37 C. prior to use and emulsify with a high shear mill. After emulsification is complete add the coconut oil (Spectrum Chemicals, U.S.) and the glycerin (Spectrum chemicals, U.S.) and emulsify as previous. Check for stability of emulsification at this point. After a 30 minute stability has been achieved add the part “C” slowly with rapid stirring. When the aloe is fully incorporated into the mixture add the previously made part “B” and re-emulsify in colloid mill. Allow this compound to rest for 30 minutes and then add the previously prepared part “D” and pass through homogenizer/emulsifier mixer again. Add the previously treated/incubated ceramide penta-peptide and the collagen and homozinize these ingredients into the previously made formulation. Allow final product to return to its normal Theological state for 30 minutes prior to using or packaging.

Final product can be bulk stored in sealed containers at 4 C. for several weeks but must be remixed prior to filling.

This composition has been shown to be stable at room temperature (20° C.) for at least 1 year.

The product is used by applying to the skin a small amount and gently rubbing it into the treated area and then allowing it to absorb into the skin. A once a day application has shown to be effective in reducing the effect of wrinkles and to improve skin health.

The following examples demonstrate the best mode that has been obtained to date for passive delivery of high molecular weight substances in what appears to be a non-inflammatory composition for the compounds being tested.

### EXAMPLE 1

The following example of optimized formulation for ceramide was compounded at room temperature by using the steps shown:

1. Pre-preparation of Part “A” consists of dissolving the PVP K-40 slowly with high shear mixing into 95% ethyl alcohol to make a 25% (w/v) solution. The mixture is allowed to “rest” for 48 hours to assure all the polymer is swollen and the solution is complete.

2. Base ingredient “B” is pre-made by dissolving with high shear mixing the Kelgin (sodium alginate) in distilled water to achieve a final concentration of 3% (w/v). Add to this a preservative, methylparaben, in a 1% concentration to prevent bacterial and mold growth in both the stock material and the finished product. This mixture must be allowed to achieve complete dissolution and to age for a minimum of 48 hours before use for proper functionality.

3. Pre-prep ingredient “C” is hyaluronic acid powder that is dissolved in a natural aloe vera plant extract so as to achieve a final concentration of 0.5% of the calculated final production lot size. This must be done using high shear mixing while maintaining a temperature of 4-10° C. This mixture must incubate for 24 hours to reach stabilization.

4. Part “D” in the pre-manufacture protocol is comprised of the following ingredients that are blended together with high shear mixing to produce a smooth uniform mixture blend: purified water (65%), stearyl alcohol (14%), cetyl esters wax (3.5%), glyceryl monosterate (2%), polyoxyethylene stearyl ether (3%), sorbitol (10%), isopropyl palmitate (2%), methylparaben (0.16%), propylparaben (0.4%), captan (0.5%). Part “D” is blended at a temperature of 60° C. and then cooled to room temperature for storage and use.

5. Activation/incubation of the active ingredients is the next step in the production of the finished product. This is accomplished by adding to part “B” the Ceramide penta-peptide mixture selected and the collagen using high shear emulsification. The mixture is then allowed to age for 48 hours at room temperature to enable the binding of the peptides with the polysaccharides in preparation for the final blending and solvent replacement steps.

6. Proceed to combine part “A” with part “C” and the coconut oil, glycerin with a homogenizing mill.

7. Add the part “D” to product of 6 and remix.

8. Let product 7 rest for 30 minutes and then package use.

The composition of all the components in the above formulation in their combined form resulted in the composition as is shown on Table 1. This composition contained a theoretical amount of 13% by weight of mixed penta-peptides. The resulting cream was tested in human studies. The human studies demonstrated the ability of the formulation to reduce the shortly after application of the appearance of fine lines and wrinkles on the test subjects skin without any irritation.

TABLE 1
Composition of Intradermal Formulation Ingredient
grams % of Final Formulation:

| Formula | % |
|---|---|
| PVP Base | 16 |
| Kelgin HV | |
| base | 10 |
| Coco | 8 |
| Glycerine | 7.5 |
| Aloe | 20 |
| VC | 10 |
| SK-penta | |
| peptide | 13 |
| K-Oil | 11 |
| Collagen | 4 |
| Hyaluronic | |
| acid | 0.5 |

Prior to testing in humans, the above formulation without the actives was evaluated to determine if it would produce allergic skin reactions following epicutaneous application to albino guinea pigs, otherwise known as the Buehler Sensitization Test. The study was undertaken by Toxicon Corporation, Bedford, Mass. under study #00-2745-G2. The conclusions of the study indicated that the above formulation is not considered to be a skin sensitizer since none of the test animals exhibited erythema and/or edema at the challenge exposure (36 hours) following an induction phase (6 hours/day; 5 days/week, 3 consecutive weeks).

In another study, Toxicon performed an acute toxicity in Rabbits-45 hours, under study #00-2745-G1. Assessments including clinical observations and body weight measurement, hematological and clinical chemistry status, necropsy and organ weight determinations, and histopathological analysis of selected tissues. The results indicated that the intradermal product did not elicit any acute toxicity at a dose of 5 grams/animal, as evidenced by the lack of any significant differences in any of the assessed parameters compared to the control animals.

A clinical study is being conducted on the formulation by Dr. Jay Ellenby of the University of Miami Jackson Memorial Medical Center. The experimental protocol is being used to verify the preferred embodiment of this invention so it will function as desired. The study is expected to show conclusively that the formulation is well tolerated, and no adverse events are attributable to the, application or treatment.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the claims appended hereto.

What is claimed is:

1. A composition of matter for intradermal delivery of an active substance comprising:
the active substance in combination with a biopolymer, which combination is linked to a base formulation whereby the base formulation aids in the intradermal delivery of the active substance.

2. The composition of claim 1 in which the biopolymer is selected from the polymer group consisting of polyvinylpyrroliodone, alginates, chitin, collagen, and elastin.

3. The composition of claim 1 in which the biopolymer is preferably polyvinylpyrroliodone.

4. The composition of claim 1 in which the base formulation includes an ingredient that minimizes skin inflammation.

5. The composition of claim 1 wherein the base formulation further includes at least one of natural herbal, vegetable and animal products.

6. The composition of claim 5 in which the vegetable product is selected from the group consisting of natural extract of marcrocystis sp., oil of coconut, corn oil, soy oil, and almond oil.

7. The composition of claim 5 in which the animal product is selected from the group consisting of glycerol and oil from avian species such as emu, chicken, turkey, and ostrich.

8. The composition of claim 5 in which the herbal product is aloe vera.

9. The composition of claim 1 wherein the base formulation includes solvents selected from the group consisting of aqueous, non-aqueous, polar, and non-polar which are combined to form a homogeneous mixture with adjustable viscosity.

10. The composition of claim 1 wherein the base formulation further includes at least one solvent selected from the group consisting of ethyl alcohol, isopropyl alcohol, acetone, and methanol.

11. The composition of claim 1 which facilitates intradermal delivery of ceruimide peptides without skin irritation.

12. A method for enhancing intradermal delivery of an active substance comprising:
combining the active substance with a biopolymer;
creating a base formulation that includes a least one ingredient that minimizes skin inflammation;
linking the biopolymer and active substance combination with the base formulation to form a composition of matter; and
applying the composition of matter topically to a patient.

13. The method of claim 12 in which the step of combining the active substance with a biopolymer includes a pre-use incubation phase wherein the biopolymer acts as a binding/graphing agent to enhance intradermal delivery of the active substance after the active substance and biopoolymer are linked with the base formulation.

14. The method of claim 12 in which the step of linking the biopolymer and active substance combination with the base formulation to form a composition of matter further comprises:
adding the biopolymer and active substance combination to a solvent rich base formulation to create a mixture; and
homogenizing the mixture during which the solvent is removed.

15. The method of claim 12 in which the step of applying the composition of matter topically to a patient includes at least one of use of a intradermal patch, cream, lotion, balm, gel, rub and ointment.

16. The method of claim 12 wherein the composition of matter includes a biopolymer, and at least one of natural herbal, vegetable and animal products.

17. The method of claim 12 wherein the composition of matter is adjusted for a hydrophobic/lipophobic nature of the composition to allow integration of substances with various solubility characteristics.

18. The method of claim 12 wherein the base formulation includes solvents selected from the group consisting of aqueous, non-aqueous, polar, and non-polar which are combined to form a homogeneous mixture with adjustable viscosity.

19. The method of claim 12 wherein an organic solvent of high volatility selected from the group consisting of ethyl alcohol, isopropyl alcohol, acetone, and methanol is used in preparing the base formulation.

20. The method of claim 12 which facilitates intradermal delivery of ceruimide peptides without skin irritation.
